(19) United States
(12) Patent Application Publication
(10) Pub. No.: US 2018/0021278 A1
(43) Pub. Date: Jan. 25, 2018

(54) TREATMENT OR PREVENTION OF INFLAMMATION USING SERINE

(71) Applicant: NESTEC S.A., Vevey (CH)

(72) Inventors: Magali Faure, Forel (CH); Stephanie Blum-Sperisen, Pully (CH); Mohamed Nabil Bosco, Epalinges (CH); Viral Brahmbhatt, Guragon, Haryana (IN); Denis Breuille, Lausanne (CH)

(21) Appl. No.: 15/544,073

(22) PCT Filed: Jan. 22, 2016
(86) PCT No.: PCT/EP2016/051279

§ 371 Date: Jul. 17, 2017

(30) Foreign Application Priority Data
Jan. 23, 2015 (EP) 15152317.2

(51) Int. Cl. A61K 31/198; A61K 45/06

(52) U.S. Cl. CPC A61K 31/198; A61K 45/06

(57) ABSTRACT

Methods and compositions are provided that treat or prevent inflammation using serine. A therapeutically effective amount of serine is administered to an individual, for example a human or other mammal, that has inflammation or is at risk of inflammation. The methods and compositions can control and/or alleviate an inflammatory reaction of the body, such as colitis.

### BACKGROUND

The present disclosure generally relates to health and nutrition. More specifically, the present disclosure relates to methods and compositions for treatment or prevention of inflammation using serine.

Inflammation is a complex reaction of the innate immune system that involves the accumulation and activation of leucocytes and plasma protein at sites of infection, toxin exposure, or cell injury. Although inflammation has a protective function in controlling infections and promoting tissue repair, inflammation can also cause tissue damage and disease. Gastrointestinal diseases such as inflammatory bowel disease (for example, Crohn's disease, ulcerative colitis, and pouchitis), food allergies, and atopic dermatitis resulting from food allergies are always accompanied by aberrant intestinal inflammatory responses at different levels. The alleviation of this intestinal inflammation by balancing pro- and anti-inflammatory cytokines or induction of regulatory cytokines has been suggested as a possible treatment for these chronic diseases. There are numerous such cytokines of which IFN-γ, IL1, IL8, IL12 and TNF-α, for example, are regarded as pro-inflammatory and IL10 and TGF-β, for example, are regarded as anti-inflammatory.

Unwanted inflammation can be treated by proper medication. However, medication can result in undesirable side effects and often requires the supervision of medical personnel. Some nutritional interventions, such as with n-3 poly-unsaturated fatty acid (PUFA), achieve diminished inflammatory cell functions but also decrease cell-mediated immune response (e.g. lymphocyte proliferation and NK activity), which can lead to potential detrimental effects with regard to the host defense.

### SUMMARY

The present disclosure provides methods for treatment or prevention of inflammation comprising administering serine to an individual in need thereof or at risk thereof To the best knowledge of the inventors, no clear link has ever been made to date between serine and inflammation. The inventors surprisingly found that supplementation of the diet with serine reduced colitis in a rat model.

Accordingly, in a general embodiment, a method for treating inflammation is provided. The method comprises administering to an individual having inflammation a composition comprising a therapeutically effective amount of serine. In another embodiment, a method for preventing inflammation is provided. The method comprises administering to an individual at risk thereof a composition comprising a therapeutically effective amount of serine. In yet another embodiment, a method for treating or preventing colitis is provided. The method comprises administering to an individual in need thereof or at risk thereof a composition comprising a therapeutically effective amount of serine. The colitis can be acute colitis and/or chronic colitis. In yet another embodiment, a method for treating inflammatory Bowel Disease (IBD) is provided. The method comprises administering to an individual in need thereof a composition comprising a therapeutically effective amount of serine. In yet another embodiment, a method for preventing or postponing relapse in an IBD patient is provided. The method comprises administering to an individual in need thereof a composition comprising a therapeutically effective amount of serine. The IBD may be Crohn's Disease or Ulcerative Colitis.

In an embodiment, the composition is administered by a route selected from the group consisting of oral, topical, enteral and parenteral. In an embodiment the composition is a medical food. In an embodiment, the composition is in a form selected from the group consisting of a nutritionally complete product, a drink, a dietary supplement, a meal replacement, a food additive, a supplement to a food product a powder for dissolution, an enteral nutrition product, an infant formula, and combinations thereof In an embodiment, the composition is in a form selected from the group consisting of a dairy product, a chilled beverage, a shelf-stable beverage, a soup, a nutritional bar, a confectionery, a milk product, a fermented milk product, a yogurt, a milk-based powder, a puree, a cereal product, a fermented cereal-based product, an ice-cream, a candy, a biscuit, a cake, a chocolate, a cappuccino, a coffee, a pet food, a pet beverage and combinations thereof

In another embodiment, the composition is a medicament.

In an embodiment, the inflammation is selected from the group consisting of acute inflammation, skin inflammation, inflammatory bowel syndrome, liver inflammation, allergy, atopy, bone inflammation, rheumatoid arthritis, systemic lupus, Gougerot-Sjogren's syndrome, Reiter's syndrome, poliomyelitis, dermato-myositis, thyroiditis, Basedow, Hashimoto, type I diabetes, Addison's disease, auto-immunes hepatitis, celiac disease, Biermer's disease, multiple sclerosis, myasthenia, encephalomyelitis, eye inflammation, obesity-associated inflammation, age-related low-grade inflammation, Blau's syndrome, Alzheimer's disease, cardiovascular diseases, atherosclerosis, metabolic syndrome, gingivitis, paronditis, and combinations thereof.

In an embodiment, the individual is selected from the group consisting of an infant, a child, an adolescent, an adult and an elderly person. In a preferred embodiment, the individual is selected from the group consisting of an adult and elderly person.

In an embodiment, the nutritional composition further comprises at least one component selected from the group consisting of a prebiotic, a probiotic, a synbiotic, an additional amino acid, a protein, a nucleotide, a fish oil, a non-marine source of omega-3 fatty acids, a phytonutrient, an antioxidant, and combinations thereof.

In an embodiment, the composition is administered in an amount to provide about 0.07 to about 0.35 g of serine/kg of body weight of the individual per day.

In another embodiment, a method of making a composition for treating or preventing inflammation is provided. The method comprises adding serine to a foodstuff to form a serine-enriched foodstuff.

An advantage of the present disclosure is to provide methods of treating or preventing inflammation and provide compositions useful in such methods.

Another advantage of the present disclosure is to reduce or prevent inflammation by oral administration of a therapeutic nutritional composition or medicament incorporating serine.

Yet another advantage of the present disclosure is to reduce or prevent inflammation using a natural compound that has anti-inflammatory properties and that does not have any detrimental effects with regard to the subject's immune defense.

Still another advantage of the present disclosure is to reduce or prevent inflammation more safely than known medication.

Another advantage of the present disclosure is to reduce or prevent inflammation with tolerable side effects or no side effects.

Additional features and advantages are described herein, and will be apparent from, the following Detailed Description and the Figures.

### BRIEF DESCRIPTION OF THE FIGURES

FIG. 1 depicts the chemical structure of serine.

FIG. 2 is a graph of experimental results from the example disclosed herein.

### DETAILED DESCRIPTION

As used in this disclosure and the appended claims, the singular forms “a,” “an” and “the” include plural referents unless the context clearly dictates otherwise. Thus, for example, reference to “an amino acid” or “the amino acid” includes two or more amino acids. The term “and/or” used in the context of “X and/or Y” should be interpreted as “X,” or “Y,” or “X and Y.” Where used herein, the term “example,” particularly when followed by a listing of terms, is merely exemplary and illustrative, and should not be deemed to be exclusive or comprehensive.

As used herein, “about” is understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

All percentages expressed herein are by weight of the total weight of the composition unless expressed otherwise. When reference is made to the pH, values correspond to pH measured at 25° C. with standard equipment.

The terms “condition” and “disorder” mean any disease, condition, symptom, or indication. As used herein, an “effective amount” is an amount that prevents a deficiency, treats a condition or disorder in an individual or, more generally, reduces symptoms, manages progression of the condition or disorder or provides a nutritional, physiological, or medical benefit to the individual.

The terms “treatment” and “treating” include any effect that results in the improvement of the condition or disorder, for example lessening, reducing, modulating, or eliminating the condition or disorder. Non-limiting examples of “treating” or “treatment of” a condition or disorder include: (1) inhibiting the condition or disorder, i.e. arresting the development of the condition or disorder or its clinical symptoms and (2) relieving the condition or disorder, i.e. causing the temporary or permanent regression of the condition or disorder or its clinical symptoms. The terms “treating” and “treatment” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms do not necessarily imply that a subject is treated until total recovery. A treatment can be patient- or doctor-related.

The terms “prevention” or “preventing” mean causing the clinical symptoms of the referenced condition or disorder to not develop in an individual that may be exposed or predisposed to the condition or disorder but does not yet experience or display symptoms of the condition or disorder. “Prevention” includes reduction of risk and/or severity of a condition or disorder.

“Animal” includes, but is not limited to, mammals, which includes but is not limited to, rodents, aquatic mammals, domestic animals such as dogs and cats, and farm animals such as sheep, pigs, cows and horses, and humans. Where “animal,” “mammal” or a plural thereof is used, these terms also apply to any animal that is capable of the effect exhibited or intended to be exhibited by the context of the passage. As used herein, the terms “patient” and “individual” are understood to include an animal, especially a mammal, and more especially a human that is receiving or intended to receive treatment, as treatment is herein defined. While the terms “individual” and “patient” are often used herein to refer to a human, the present disclosure is not so limited. Accordingly, the terms “individual” and “patient” refer to any animal, mammal or human that can benefit from the treatment.

An animal is considered “elderly” if it has surpassed the first two thirds of the average expected lifespan in its country of origin, preferably if it has surpassed the first three quarters of the average expected lifespan in its country of origin, more preferably if it has surpassed the first four fifths of the average expected lifespan in its country of origin. An “elderly human” means a person with a chronological age of 65 years or older.

As used herein, “long term administrations” are continuous administrations (e.g. at least twice a week, preferably daily) for 6 weeks or more. “Short term administrations” are continuous administrations (e.g. at least twice a week, preferably daily) for less than 6 weeks.

The terms “food,” “food product” and “food composition” mean a product or composition that is intended for ingestion by a human and provides at least one nutrient to the human The compositions disclosed herein may lack any element that is not specifically disclosed herein. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified. Any embodiment disclosed herein can be combined with any other embodiment disclosed herein.

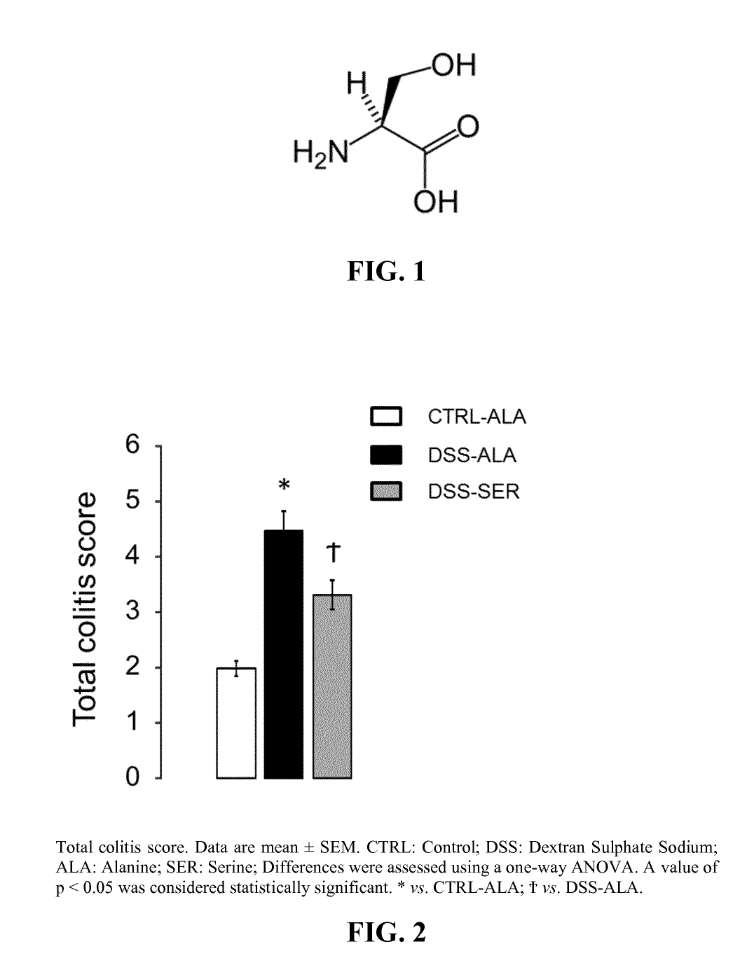

Referring to the figures, FIG. 1 shows the chemical structure of serine. Serine is a non-aromatic hydroxyl, polar (no charge) amino acid. Serine is classified as a non-essential amino acid for mammals because it can be synthesized endogenously from essential amino acids or from other complex nitrogenous sources.

In an aspect of the present disclosure, a method of treating inflammation is provided. The method comprises administering a composition comprising a therapeutically effective amount of serine to an individual having the inflammation. In another aspect of the present disclosure, a method of preventing inflammation is provided. The method comprises administering a therapeutically effective amount of serine to an individual at risk thereof In yet another aspect of the present disclosure, a method of controlling and/or alleviating an inflammatory reaction of the body is provided. The method comprises administering a therapeutically effective amount of serine to an individual having the inflammatory reaction of the body.

The composition may be administered to humans or animals such as companion animals, pets or livestock. In an embodiment, the composition is administered in an amount to provide about 0.07 to about 0.35 g of serine/kg of body weight per day. The composition has beneficial effects for any age group. Preferably, the composition is intended for infants, juveniles, adults or elderly. However, the composition can be administered to mothers during pregnancy and/or lactation to treat the infant. The composition can be administered to the individual for a short-term administration or a long-term-administration. Preferably the composition is administered enterally, for example orally.

Non-limiting examples of inflammatory conditions that may be treated or prevented by the methods and compositions disclosed herein include but are not limited to acute inflammations such as sepsis, infections, burns and chronic inflammations such as inflammatory bowel disease, Crohn's disease, ulcerative colitis, inflammatory bowel syndrome, necrotizing enterocolitis, skin inflammation, such as UV or chemical-induced skin inflammation, eczema, reactive skin, psoriasis, vitiligo, acne, liver inflammation, alcoholic cirrhosis, allergy, atopy, bone inflammation, rheumatoid arthritis, systemic lupus, Gougerot-Sjogren's syndrome, Reiter's syndrome, poliomyelitis, dermato-myositis, thyroiditis, Basedow, Hashimoto, type I diabetes, Addison's disease, auto-immunes hepatitis, celiac disease, Biermer's disease, multiple sclerosis, myasthenia, encephalomyelitis, eye inflammation, obesity-associated inflammation, age-related low-grade inflammation, Blau's syndrome, Alzheimer's disease, cardiovascular diseases, atherosclerosis, metabolic syndrome, gingivitis, paronditis and combinations thereof.

In an embodiment, the composition contains an additional amino acid selected from the group consisting of alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, and combinations thereof In an embodiment, the composition may contain additionally an amino acid precursor. In one embodiment the composition contains an amino acid precursor selected from the cysteine precursors cystathionine, N-acethycysteine and/or DACE. In another embodiment, the serine is the only amino acid in the composition.

The serine in the composition may be in free form (i.e. a monomer) or may be part of a dipeptide, a tripeptide, or a polypeptide (e.g. a protein, which as used herein means a polypeptide having 20 or more amino acids).

The composition may be a food product, a supplement to a food product, an animal food product, or a pharmaceutical composition. For example, the product may be a nutritional composition, a nutraceutical, a drink, a food additive or a medicament. A food additive or a medicament may be in the form of tablets, capsules, pastilles or a liquid for example. Food additives or medicaments are preferably provided as sustained release formulations, allowing a constant supply of serine for a prolonged time.

The composition may be a medical food. A medical food product is specially formulated and intended for the dietary management of diseases or medical conditions (e.g., prevent or treat diseases or undesirable medical conditions). A medical food product can provide clinical nutrition, for example fulfilling special nutritional needs of patients with a medical condition or other persons with specific nutritional needs. A medical food product can be in the form of a complete meal, part of a meal, as a food additive, or a powder for dissolution.

In an embodiment, the nutritional compositions are in a form selected from the group consisting of tablets, capsules, liquids, chewables, soft gels, sachets, powders, syrups, liquid suspensions, emulsions, solutions, or combinations thereof. In an embodiment, the nutritional compositions are oral nutritional supplements. Alternatively, the nutritional compositions may be tube feedings.

The composition can provide complete nutrition or incomplete nutrition. Complete nutrition provides types and levels of macronutrients (protein, fats and carbohydrates) and micronutrients to be sufficient to be a sole source of nutrition for the animal to which it is being administered. Patients can receive 100% of their nutritional requirements from such complete nutritional compositions. Incomplete nutrition does not provide levels of macronutrients (protein, fats and carbohydrates) or micronutrients to be sufficient to be a sole source of nutrition for the animal to which it is being administered. A partial or incomplete nutritional composition is preferably used as a nutritional supplement.

The composition is preferably selected from the group consisting of milk powder based products; instant drinks; ready-to-drink formulations; nutritional powders; nutritional liquids; milk-based products, in particular yoghurts or ice cream; cereal products; beverages; water; coffee; cappuccino; malt drinks; chocolate flavored drinks; culinary products; soups; tablets; and syrups. Milk may be any milk obtainable from animal or plant sources and is preferably cow's milk, human milk, sheep milk, goat milk, horse milk, camel milk, rice milk or soy milk. Additionally or alternatively, milk-derived protein fractions or colostrum may be used.

The composition may comprise protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional pharmaceutical additives and adjuvants, excipients and diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. Further, the composition may contain an organic or inorganic carrier material suitable for oral or enteral administration as well as vitamins, minerals trace elements and other micronutrients in accordance with the recommendations of government bodies.

The composition may comprise a protein source, a carbohydrate source and/or a lipid source. Any suitable protein source may be used, for example animal proteins (such as milk proteins, meat proteins and egg proteins), vegetable proteins (such as soy protein, wheat protein, rice protein, and pea protein), mixtures of free amino acids, or combinations thereof Milk proteins, such as casein and whey, and soy proteins are particularly preferred.

If the composition includes a fat source, the fat source preferably provides 5% to 50% of the energy of the composition, preferably 10% to 40%, more preferably 20% to 30% of the energy. Vegetable fats such as soy oil, palm oil, coconut oil, safflower oil, sunflower oil, corn oil, canola oil, and lecithins are particularly suitable. Animal fats such as milk fat may be included if desired.

A source of carbohydrates may provide 20% to 80% of the energy of the composition, preferably 30% to 70% of the energy of the composition. Any suitable carbohydrate may be used, for example sucrose, lactose, glucose, fructose, corn syrup solids, maltodextrins, and mixtures thereof. Dietary fiber may also be added if desired. The dietary fiber may be from any suitable origin, including for example soy, pea, oat, pectin, guar gum, gum Arabic, fructooligosaccharides, galacto-oligosaccharides, sialyl-lactose and oligosaccharides derived from animal milks.

Suitable vitamins and minerals may be included in the composition. The presence and amounts of specific vitamins and minerals will vary depending on the intended recipient of administration.

In an embodiment, the composition further comprises one or more nucleotides, synbiotics, fish oils, non-marine sources of omega-3 fatty acids, phospholipids, phytonutrients and/or antioxidants. As used herein, a synbiotic is a combination of a prebiotic and a probiotic that synergistically improves the microflora of the intestine. Non-limiting examples of suitable fish oils include fish oils providing docosahexaenoic acid (DHA) and eicosapentaenoic acid (EPA). Non-limiting examples of suitable phytonutrients include quercetin, curcumin and limonin. Antioxidants are molecules capable of slowing or preventing the oxidation of other molecules. Non-limiting examples of suitable antioxidants include vitamin A, carotenoids, vitamin C, vitamin E, selenium, flavonoids, Lactowolfberry, Goji (wolfberry), polyphenols, lycopene, lutein, lignan, coenzyme Q10 (CoQ10), hesperidine and glutathione. Non-limiting examples of phospholipids include phosphatidylcholine, phosphatidylserine and phosphatidylethanolamine.

In another aspect of the present disclosure, a method of making a composition for treatment or prevention of inflammation is provided. The method comprises adding serine to a foodstuff to form a serine-enriched foodstuff.

### EXAMPLE

The following non-limiting example is illustrative of treatment or prevention of inflammation using serine according to the present disclosure.

### Example 1

The experimental procedures were carried out in accordance to European guidelines for the care and use of laboratory animals (Directive 2010/63/UE). 30 male Sprague-Dawley rats from Janvier (France), aged 6-8 months and weighting around 500/700g on the day of arrival, were used for this study. Animals were individually housed in cages. During the study, they had free access to food and drinking water or a dextran sulfate sodium (DSS, MW 36-44 kDa, ICN Biomedicals) solution ad libitum. Colitis was induced by treating rats with 5% DSS from D0 to D8 (acute colitis) and then 2% DSS from D9 to D28 (chronic colitis). DSS was dissolved in autoclaved water and provided ad libitum to the rats. Animals of the control group were given water not containing DSS from D0 to D28.

3 groups of rats received the following treatment and diets:

Group CTRL (CTRL-ALA, n=10) received the control diet, a dry semisynthetic powder consisting of (g/kg): carbohydrates 646 (wheat starch), proteins 120 (supplied by herring meal balanced to meet all amino acid requirements), lipids 64 (groundnut oil 45, sunflower oil 10, rapeseed oil 9), agar-agar 30, mineral mix 70 (UAR 205b: CaHPO4, 30.1; KCl, 7; NaCl, 7; MgO, 0.735; MgSO4, 3.5; Fe2O3, 0.21; FeSO47H2O, 0.35) and vitamin mix 10 [UAR 200]. The control diet was isonitrogenous with other diets through supplementation with alanine (10 g/kg dry matter). The serine concentration of the control diet was 5 g/kg dry matter.

Group DSS control (DSS-ALA, n=10) received the control diet supplemented with alanine (10 g/kg dry matter). The serine concentration of the control diet was 5 g/kg dry matter.

Group DSS (DSS-SER, n=10) received the control diet supplemented with 10 g/kg (dry matter) serine.

During an adaptation period of 8 d, rats from each group were fed their respective diets. From D0, they received or not DSS in their drinking water as described before.

At the end of the experiment (D28), animals were anesthetized using a combination of ketamine and xylasine and then euthanized by cervical dislocation. As soon as animals were euthanized, an abdominal midline incision was performed and the colon was collected from the colocecal junction to the anal verge. The colon was rinsed, small pieces of colon from both the proximal, median and distal colon were collected and placed in refrigerated 4% formalin. Samples were dehydrated and then embedded in wax in order to obtain transversal sections. Sections were used for Hematoxylin-eosin (HE) staining in order to assess mucosal degeneration, mucosal regeneration and hyperplasia, acute and sub-acute inflammation. A score (ranging from 0 to 3) was finally determined for each criterion on each colon section and a total score was calculated.

Total scores are presented in FIG. 2. Total colitis was significantly increased in the DSS-ALA group as compared to CTRL (p<0.001). It was significantly lower in the DSS group who received a supplementation in Serine (DSS-SER) than in the DSS-ALA group (p<0.05).

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for treating inflammation comprising administering to an individual having inflammation a composition comprising a therapeutically effective amount of serine.

2. The method of claim 1 wherein the composition is administered by a route selected from the group consisting of oral, topical, enteral and parenteral.

3. The method of claim 1, wherein the composition is in a form selected from the group consisting of a nutritionally complete product, a drink, a dietary supplement, a meal replacement, a food additive, a supplement to a food product a powder for dissolution, an enteral nutrition product, an infant formula, and combinations thereof.

4. The method of claim 1, wherein the inflammation is selected from the group consisting of acute inflammation, skin inflammation, inflammatory bowel disease including crohn's disease and/or ulcerative colitis, inflammatory bowel syndrome, liver inflammation, allergy, atopy, bone inflammation, rheumatoid arthritis, systemic lupus, Gougerot-Sjogren's syndrome, Reiter's syndrome, poliomyelitis, dermato-myositis, thyroiditis, Basedow, Hashimoto, type I diabetes, Addison's disease, auto-immunes hepatitis, celiac disease, Biermer's disease, multiple sclerosis, myasthenia, encephalomyelitis, eye inflammation, obesity-associated inflammation, age-related low-grade inflammation, Blau's syndrome, Alzheimer's disease, cardiovascular diseases, atherosclerosis, metabolic syndrome, gingivitis, paronditis, and combinations thereof.

5. The method of claim 1, wherein the individual is selected from the group consisting of an infant, a child, an adolescent, an adult and an elderly person.

6. The method of claim 1, wherein the nutritional composition further comprises at least one component selected from the group consisting of a prebiotic, a probiotic, a synbiotic, an additional amino acid, a protein, a nucleotide, a fish oil, a non-marine source of omega-3 fatty acids, a phytonutrient, an antioxidant, and combinations thereof.

7. The method of claim 1, wherein the composition is administered in an amount to provide about 0.07 to about 0.35 g of serine/kg of body weight of the individual per day.

8. A method for preventing inflammation comprising administering to an individual at risk thereof a composition comprising a therapeutically effective amount of serine.

9. The method of claim 8 wherein the composition is administered by a route selected from the group consisting of oral, topical, enteral and parenteral.

10. The method of claim 8, wherein the composition is in a form selected from the group consisting of a nutritionally complete product, a drink, a dietary supplement, a meal replacement, a food additive, a supplement to a food product a powder for dissolution, an enteral nutrition product, an infant formula, and combinations thereof.

11. The method of claim 8, wherein the inflammation is selected from the group consisting of acute inflammation, skin inflammation, inflammatory bowel disease including crohn's disease and/or ulcerative colitis, inflammatory bowel syndrome, liver inflammation, allergy, atopy, bone inflammation, rheumatoid arthritis, systemic lupus, Gougerot-Sjogren's syndrome, Reiter's syndrome, poliomyelitis, dermato-myositis, thyroiditis, Basedow, Hashimoto, type I diabetes, Addison's disease, auto-immunes hepatitis, celiac disease, Biermer's disease, multiple sclerosis, myasthenia, encephalomyelitis, eye inflammation, obesity-associated inflammation, age-related low-grade inflammation, Blau's syndrome, Alzheimer's disease, cardiovascular diseases, atherosclerosis, metabolic syndrome, gingivitis, paronditis, and combinations thereof.

12. The method of claim 8, wherein the individual is selected from the group consisting of an infant, a child, an adolescent, an adult and an elderly person.

13. The method of claim 8, wherein the nutritional composition further comprises at least one component selected from the group consisting of a prebiotic, a probiotic, a synbiotic, an additional amino acid, a protein, a nucleotide, a fish oil, a non-marine source of omega-3 fatty acids, a phytonutrient, an antioxidant, and combinations thereof.

14. The method of claim 8, wherein the composition is administered in an amount to provide about 0.07 to about 0.35 g of serine/kg of body weight of the individual per day.

15. (canceled)

16. A method for treating or preventing IBD, comprising administering to an individual in need thereof or at risk thereof a composition comprising a therapeutically effective amount of serine.

17. (canceled)

18. A method according to claim 16, wherein the IBD is Crohn's disease.

19. A method according to claim 16, wherein the IBD is Ulcerative colitis.

20-22. (canceled)
